(19) United States
(12) Patent Application Publication
(10) Pub. No.: US 2022/0265866 A1
(43) Pub. Date: Aug. 25, 2022

(54) METHODS FOR ANALYZING TUMOR INNATE IMMUNE INTERACTIONS USING THE ZEBRAFISH XENOGRAFT MODEL AS A LIVING BIOMARKER

(71) Applicant: Fundaçâo D. Anna de Sommer Champalimaud e Dr. Carlos Montez Champalimaud, Lisboa (PT)

(72) Inventors: Rita Leonor ALVARES CABRAL DE FIGUEIREDO FIOR SOUSA SOARES, Lisboa (PT); Vanda Cristina BARROSO PÓVOA, Amora (PT)

(21) Appl. No.: 17/672,254

(22) Filed: Feb. 15, 2022

Related U.S. Application Data
(60) Provisional application No. 63/151,408, filed on Feb. 19, 2021.

(51) Int. Cl. A61K 49/00; A01K 67/027; G01N 33/50

(52) U.S. Cl. CPC A61K 49/0058; A01K 67/0271; G01N 33/5088; A01K 2227/40

(57) ABSTRACT

The present invention discloses methods of analyzing the immune reactive status of human tumors using zebrafish xenografts.

By analyzing the engraftment/clearance profiles in zebrafish xenografts, the present invention aims to identify new mechanisms of innate immune evasion/suppression and consequent biomarkers for immunotherapy; innate immunomodulator molecules that can be used in combination with established cancer immunotherapies, therefore engaging both arms of the immune system.

Further, the present invention allows the discovery of compounds with capacity to increase engraftment and, therefore, find immunomodulator molecules that can be used in transplantation procedures. Finally, this in vivo method will allow to select eligible patients for immunotherapy treatment.

The present invention claims the priority of the Provisional U.S. Patent Application No. 63/151,408, filed on Feb. 19, 2021, the contents of which are incorporated by reference herein in its entirety.

### TECHNICAL FIELD

The present invention is inserted in the field of Oncolmmunology and immunology and discloses methods for analyzing tumor innate immune interactions using zebrafish xenografts.

### BACKGROUND ART

Cancer immunoediting is a dynamic process of crosstalk between tumor cells and the immune system. Clinically detectable tumors represent the ultimate consequence of tumor immunoediting, which includes the detection and clearance of the majority of the immunogenic clones by the immune system (Schreiber et al., 2011).

Clones that escape immune detection further hijack immune cells to support tumorigenesis (Liu and Cao, 2016). Although the concept of immunoediting is well established, the role of innate immune cells in shaping and selecting subclones, as well as the mechanisms that allow for innate immune evasion remain less explored (Cortez-Retamozo, 2012).

In recent years, there has been a major effort to uncover the role of adaptive immunity on tumor immune surveillance/evasion/suppression, which has been translated into promising new immunotherapies, such as the immune checkpoint blockers (ICB) (Liu and Zeng, 2012). ICB therapies aim to remove inhibitory pathways that block anti-tumor T-cell responses in the tumor microenvironment (TME). However, immunotherapy may fail because tumor cells do not express sufficient neo-antigens (not immunogenic enough) (Gentles et al., 2015).

Another major obstacle may be the presence of a suppressive (cold) TME-composed of stroma and a variety of immune cells, such as Regulatory T cells (Treg), myeloid-derived suppressor cells (MDSC), alternative activated pro-tumoral macrophages (“M2-like”) and neutrophils (“N2-like”), that may block anti-tumor immune responses (Gentles et al., 2015 and Cassetta and Pollard, 2018). In fact, innate myeloid-derived cells effectively represent the major component of the TME in most solid tumors (Gentles et al., 2015 and Van Overmeire et al., 2014), often outweighing lymphocytes or even the tumor cells themselves.

These populations of the immune system are present in all tissues. However, their role in cancer-induced immune suppression and immunotherapy remains less explored and understood. Increasing evidence supports a crucial anti- and pro-tumorigenic role for innate immune cells (Liu and Zeng, 2012). Importantly, innate pro-tumorigenic states are highly dynamic and can be selectively reverted (Gajewski et al., 2013). This reversion opens a therapeutic opportunity to switch from an immune-suppressive (COLD) TME into immune-permissive TME (HOT). Such strategy has already started to be explored, as a combinatorial approach with ICB to increase efficacy rates by modulating the TME (Cerezo-Wallis D et al., 2020; Bejarano L et al., 2021 and DeNardo D G and Ruffell B, 2019).

However, anti-cancer screening platforms for the identification of immunomodulators rely mostly on in vitro assays (Vladimer G I et al., 2017 and Grönholm M et al, 2021) that lack the complexity of a living animal and a readout of the anti-cancer effect.

In vivo screens have major advantages, since to produce in vivo phenotypes, compounds must be absorbed, reach targets, circumvent elimination but can't be too toxic, otherwise kills the animal model. This may explain why several compounds discovered in zebrafish screens rapidly moved to mammalian models and clinical trials with reduced optimization (Rennekamp A J and Peterson R T, 2015).

The zebrafish model has emerged as a powerful tool to study tumor biology and interactions with the immune system (White et al., 2013; Roh-Johnson et al., 2017; Chapman et al., 2014 and Wyatt et al. 2017). Zebrafish have a highly conserved vertebrate innate immune system, including complement, Toll-like receptors, neutrophils and macrophages capable of phagocytic activity.

Another advantage is that the full maturation of adaptive immunity only occurs at 2-3 weeks post-fertilization (Lam et al., 2006). This offers a time window to study exclusively innate immune response in vivo, independent of the adaptive system. In addition, transparency allows for unprecedented real-time imaging of cell-cell interactions and genetic tractability enables the engineering of reporter lines and mutants (Renshaw and Trede, 2012).

In the last years we have been developing and validating the zebrafish Patient Derived Xenograft model or “zAvatars” as a screening platform for personalized medicine (Fazio M et al., 2020; Fazio M and Zon L I, 2017; Fior R et al., 2017; Costa B et al., 2020; Varanda A B et al., 2020; Costa et al., 2020 and Rebelo de Almeida C et al, 2020). This assay relies on the injection of fluorescently labelled tumor cells into 2 days post fertilization (dpf) zebrafish embryos. Tumor behavior and response to anti-cancer therapy are accessed after 4 days. Zebrafish xenografts or zAvatars offer speed, single-cell resolution, large numbers of transplants and evaluation of crucial cancer hallmarks, such as metastatic and angiogenic potentials (Fior R et al., 2017; Costa B et al., 2020; Varanda A B et al., 2020; Costa et al., 2020 and Rebelo de Almeida C et al, 2020). In our studies we demonstrated that zAvatars are good reporters to identify cytotoxicity to chemo, radio and targeted therapies, including monoclonal antibodies, such as Cetuximab (Fior R et al., 2017), Bevacizumab (Rebelo de Almeida C et al., 2020) and Olaparib-PARPi (Veranda A B et al., 2020). To validate the model we performed a direct comparison between zebrafish and mouse xenografts and successfully showed similar responses (Fior R et al., 2017). We also demonstrated the feasibility of using primary patient samples for zAvatar generation (without in vitro expansion) and provided proof-of-concept experiments showing that response to chemotherapy in zAvatars correlates with patient clinical response (Fior R et al., 2017).

Surprisingly, we recently found that zebrafish xenografts can also be used as reporters of the innate TME (POvoa V et al., 2021). In other words, we found that zebrafish xenografts can reveal whether tumors generate an anti-tumoral (immune permissive/HOT) or pro-tumoral (immune suppressive/COLD) TME.

These results opened a whole new application for the xenograft model as a discovery platform for i) mechanisms of innate immune evasion/suppression; ii) biomarkers for immunotherapy; iii) innate immunomodulator molecules that can be used in combination with established cancer immunotherapies; iv) compounds for autoimmune diseases, organ transplantation or for conditions marked by uncontrolled inflammation; and finally, v) select eligible patients for immunotherapy treatment.

### SUMMARY OF THE INVENTION

The present invention provides methods to discover several immuno and oncoimmunology applications using the zebrafish xenograft model.

The present invention provides an in vivo method to find mechanisms of innate immune evasion/suppression and biomarkers of the immune response, comprising the steps of:

a. generating zebrafish xenografts;

b. dissecting the tumors from zebrafish xenografts collected in different time points;

c. quantifying the engraftment rate;

d. analyzing and comparing human vs zebrafish cells to identify human candidate genes and zebrafish interacting partners;

e. testing the functional role of the human candidate genes identified in step (d); and

f. validating the clinical relevance of the human candidate genes as biomarkers of the immune response.

Also provided by the present invention is an in vivo method to identify innate immunomodulators to boost tumor clearance using zebrafish xenografts comprising the steps of:

a) generating zebrafish xenograft using progressor tumor cells;

b) adding compounds to the water fish;

c) quantifying the engraftment rate and selecting compounds based on a significant reduction of engraftment; and

d) validating the compounds in vivo using transgenic reporters of innate immunity and immunocompromised zebrafish to confirm modulation through tumor microenvironment.

The present invention provides an in vivo method to identify innate immune suppressor compounds for autoimmune diseases, organ transplantation or for conditions marked by uncontrolled inflammation, wherein such method comprises the steps of:

a) generating zebrafish xenograft using regressor tumor cells;

b) adding compounds to the water fish;

c) quantifying the engraftment rate and selecting the compound based on significant increase of engraftment; and

d) characterizing the tumor microenvironment modulation upon treatment with the selected compounds.

The present invention further discloses an in vivo method to use zebrafish patient-derived xenografts (zAvatars) as living biomarkers of the tumor microenvironment to select eligible patients for immunotherapy treatment, wherein the method comprises the steps of:

a) generating zebrafish patient-derived xenografts in control and immunocompromised hosts;

b) characterizing the generated tumor microenvironment by quantifying the innate immune infiltrate in different time points;

c) quantifying the engraftment rate; and

d) identifying eligible patients for immunotherapy based on their engraftment rate and pro-inflammatory (HOT) tumor microenvironment.

### BRIEF DESCRIPTION OF DRAWINGS

With the purpose of providing an understanding of the principles according to the embodiments of the present invention, reference will be made to the embodiments illustrated in the figures and the terminology used to describe them.

In any case, it must be understood that there is not the intention of limiting the scope of the present invention to the content of the figures. Any subsequent alterations or modifications of the inventive characteristics herein illustrated, as well as any additional applications of the principles and embodiments of the illustrated invention, which would normally occur to a person skilled in the art, and having this description, must be considered as being within the scope of the claimed invention.

FIG. 1 shows (a) the engraftment of human cancer cells in zebrafish at 4 days post injection (dpi), with different colorectal (CRC) and breast cancer cell lines and (b) the engraftment of SW480 and zebrafish patient-derived xenografts (zPDX-zAvatars) at 4 dpi, treated with chemotherapy FOLFOX (FO) and radiotherapy (RAD) and their respective controls.

FIG. 2 shows that proliferation and cell death are not major factors for the engraftment/clearance phenotype, wherein (a-b′) show the quantification of proliferation and apoptosis in triple negative breast cancer cell lines in absolute numbers (a) and % (a′) at 4 dpi and in absolute numbers (b) and % (b′) of activated Caspase3 at 4 dpi and (c-d′) show the quantification of proliferation and apoptosis in CRC cell lines in absolute numbers (c) and % (c′) at 1 dpi and 4 dpi and in absolute numbers (d) and % (d′) of activated Caspase3 at 1 dpi and 4 dpi.

FIG. 3 shows that CRC isogenic xenografts can generate different TMEs according to their regressor/progressor phenotype, wherein (a, b) show representative confocal images of neutrophils in SW480, SW620 and MIX tumors from Tg(mpx:eGFP) zebrafish xenografts at 4 dpi; (c, d) show the quantification of neutrophils percentage within SW480, SW620 and MIX TME at 1 dpi and 4 dpi; (e, f) show representative confocal images of macrophages in SW480, SW620 and MIX tumors from zebrafish xenografts at 4 dpi and (g, h) show the quantification of macrophage percentage within SW480, SW620, and MIX tumors at 1 dpi and 4 dpi, wherein SW480 are represented in red, SW620 are represented in green, neutrophils are represented in white and macrophages are represented in white fake colors.

FIG. 4 shows that innate cell recruitment is not totally dependent on the number of injected cells by the linear regression analysis between SW480 and SW620 tumor cells number and respective innate immune cell infiltrate (neutrophils and macrophages).

FIG. 5 shows that CRC isogenic xenografts with different regressor/progressor phenotypes can generate different inflammatory TME states; wherein (a, b) show representative confocal images of SW480, SW620 and MIX xenografts injected in Tg(mpeg1:mcherry-F, tnfa:GFP-F) at 1 and 4 dpi, wherein macrophages are represented in red, TNFa+ cells are represented in green and the overlay of macrophages and TNFa+ cells (M1-like macrophages) are represented in yellow; (c) show the proportion of M1- and M2-like macrophages in the TME at 1 and 4 dpi; and (d) show the quantification of absolute numbers of M1- and M2-like macrophages in the TME at 1 and 4 dpi.

FIG. 6 shows that CRC isogenic xenografts with different regressor/progressor phenotypes can generate different inflammatory TME states, including eliciting different phagocytic activities of the TME cells, wherein (a-b) show the quantification of absolute numbers of overall macrophages (mpeg+), M1-like macrophages (mpeg+; TNFa+), M2-like macrophages (mpeg+; TNFa−), overall TNFa+ cells and TNFa+mpeg−cells at 1 dpi (a) and 4 dpi (b) of SW480 and SW620 xenografts; (c-d″) show representative confocal images of SW480 (c-c″) and SW620 (d-d″) tumor cells (in white) and macrophages (in red), showing macrophage-tumor close interaction using Tg(mpeg1:mCherryF; tnfa:eGFPF), wherein c′-c″ and d′-d″ are higher magnification of phagocyted tumor cells of c and d, respectively and (e) show the absolute quantification of phagocytosed tumor cells by M1, M2-like macrophage and TNFa+mpeg−inflammatory cells at 1 dpi.

FIG. 7 shows that engraftment/clearance phenotype is mediated by innate immune cells, wherein (a, b) show representative confocal images of SW480 (red) and SW620 (green) xenografts in runx1w84x and csf1raj4blue (panther) mutants; (c) show the quantification of engraftment in runx1w84x and csf1raj4blue (panther) mutants and respective controls; (d) show the quantification of tumor size in runx1w84x and csf1raj4blue (panther) mutants and respective controls; (e-j) show zebrafish embryos with 2 dpf were injected simultaneously with SW480 tumor cells (in green) with PBS (control), with L-PBS or with L-Clodronate liposomes into Tg(mpeg1:mcherry) background (macrophages in red); (e-g) show representative fluorescence stereoscope images of SW480 xenografts at 1 dpi in the different conditions; (h-j) representative confocal images of SW480 xenografts at 4 dpi and (k) show the quantification of engraftment and (l) the quantification of tumor size.

FIG. 8 shows innate immunoediting in zebrafish xenografts; wherein (a) shows a schematic illustration of SW480 escaper cells selection from SW480 parental xenografted; (b) shows representative confocal images of tumoral masses of SW480 parental and SW480zEscapers xenografts at 4 dpi; (c) shows the quantification of engraftment at 4 dpi; (d) shows the quantification of tumor size at 4 dpi; (e) shows the quantification of mitotic tumor cells at 4 dpi; (f) shows the quantification of apoptotic tumor cells at 4 dpi and (g) shows the quantification of macrophage present in the TME of SW480 parental versus SW480Zesc at 4 dpi.

FIG. 9 illustrates how applying an omic approach such as single cell RNAseq is possible to identify different regressor and progressor/escapers cell clones; wherein (a) shows a schematic illustration of the design of the experiment, wherein SW480 cells were injected into 2 dpf zebrafish embryos, and at 1 and 4 dpi, tumors were dissected and processed for scRNAseq; (b) shows the relative frequencies of the cell clusters present in each library replicate; (c) shows the Uniform Manifold Approximation and Projection (UMAP), representing the relative similarity between individual cells, colored by cell cluster and divided by timepoints 1 and 4 dpi; (d) shows the heatmap representation of normalized enrichment scores (NES) of representative pathways with statistically significant enrichment in gene set enrichment analysis (GSEA) and (e) shows a schematic illustration of expansion/reduction of each cluster from 1 to 4 dpi with the most representative pathways and genes.

FIG. 10 shows that macrophages may have opposite impacts on engraftment (wherein (a) represents the number of xenografts analysed per condition minimal>60) and tumor size (wherein each dot in (b) represents a xenograft) depending on the tumor. Modulation of the TME by different tumors (c) & zAvatars of CRC primary and metastasis show different engraftment rates (minimal of 20 xenografts per condition) and elicit differential recruitment of innate immune cells (d).

FIG. 11 shows preliminary data from the FDA approved Library screen in colorectal and breast cancers (a) and (b) shows zebrafish xenografts treated for 3 consecutive days with different compounds and respective DMSO control and screened according to the presence or absence of tumors, at 4 dpi.

### DESCRIPTION OF EMBODIMENTS

The present invention relates, in a first aspect, to an in vivo method to find mechanisms of innate immune evasion/suppression and biomarkers of the immune response, comprising the steps of:

a. generating zebrafish xenografts;

b. dissecting the tumors from zebrafish xenografts collected in different time points;

c. quantifying the engraftment rate;

d. analyzing and comparing human vs zebrafish cells to identify human candidate genes and zebrafish interacting partners;

e. testing the functional role of the human candidate genes identified in step (d);

f. validating the clinical relevance of the human candidate genes as biomarkers of the immune response.

In step (a), the zebrafish xenografts are generated by injecting a plurality of cancer cells into perivitelline space (PVS) or the brain of host zebrafish embryos or larvae (from 2-3 dpf), wherein host zebrafish embryos or larvae are selected from wild-type strains and transgenic zebrafish lines.

Following, in step (b), the tumors from zebrafish xenografts are collected and dissected in different time points.

In a preferred embodiment of the invention, the dissection of the tumors from zebrafish xenografts occurs at 1 dpi (day post injection) and at the end of the assay. Therefore, the second time point depends on the duration of the assay, which may vary from 4 to 8 days.

In a preferred embodiment of the invention, a pool of approximately 100 tumors cells is collected in each time point. The tumor cells are mechanically and enzymatically dissociated and filtrated (preferably using a 70 μm strainer) and used according to the chosen Omic technology.

Then (in step c), the engraftment rate is quantified by calculating the number of xenografts with a tumor at the end of the assay (4 to 8 dpi) divided by the total number of xenografts at the end of the assay.

In step (d), the human and zebrafish cells are analyzed and compared by bioinformatic analysis of results obtained by “omics” technology, namely, the universal detection of genes (genomics), mRNA (transcriptomics), proteins (proteomics) and metabolites (metabolomics).

The analysis aims to identify the regressor and progressor tumor subclones and molecular signatures (sets of genes, proteins, genetic variants or other variables that can be used as markers) of those phenotypes, as well as the host immune cells and interacting molecules present in the tumor microenvironment in both time points.

In step (e), the human candidate genes are tested by performing gain and loss of function experiments to confirm their mechanistic function. In a preferred embodiment of the invention, the experiments are selected from, but not limited to mutagenesis, RNA interference, pharmacological inhibition and CRISPR-based gene editing.

At last, in step (f), the clinical relevance of the human candidate genes are validated by bioinformatic analysis. Their molecular signatures are searched and compared to “omics” databases to interrogate patient prognosis and to find potential biomarkers of response to stratify patients for treatment.

In a second aspect, the present invention further relates to an in vivo method to identify innate immunomodulators to boost tumor clearance using zebrafish xenografts, comprising the steps of:

a) generating zebrafish xenograft using progressor tumor cells;

b) adding compounds to the water fish;

c) quantifying the engraftment rate and selecting the compound based on significant reduction of engraftment; and

d) validating the compounds in vivo using immunocompromised zebrafish to confirm modulation through tumor microenvironment.

In step (a), the zebrafish xenografts are generated by injecting a plurality of cancer cells into perivitelline space or the brain of zebrafish embryos or larvae (2-3dpf), wherein host zebrafish embryos or larvae are selected from wild-type strains and transgenic zebrafish lines, and the progressor tumor cells are tumor cell lines with engraftment rates higher than 70%.

In step (b), the compounds added to the water fish are selected from chemicals, small molecules, drugs, antibodies, peptides, secreted proteins and mixtures thereof. The addition of the compounds start in the injection day or 24 hours post-injection and compounds are renewed daily. Treated zebrafish xenografts will be compared to zebrafish xenografts treated with vehicle (controls).

In step (c), the engraftment rate is quantified, and the compounds are selected based on a significant reduction of engraftment in relation to untreated controls (vehicle). In a preferred embodiment of the present invention, a significant reduction of engraftment is a reduction of at least 20% of engraftment, when compared to the controls.

At last, in step (d), the compounds are validated in vivo using zebrafish transgenic hosts (transgenic reporters of innate immunity) and immunocompromised zebrafish (namely, zebrafish mutants, chemicals, morpholinos and mixtures thereof) to confirm modulation through the tumor microenvironment.

The present invention further relates, in a third aspect, to an in vivo method to identify innate immune suppressor compounds for autoimmune diseases, organ transplantation or for conditions marked by uncontrolled inflammation. Such method comprises the steps of:

a) generating zebrafish xenograft using regressor tumor cells;

b) adding compounds to the water fish;

c) quantifying the engraftment rate and selecting the compound based on significant increase of engraftment; and

d) characterizing the tumor microenvironment modulation upon treatment with the selected compounds.

In step (a), the zebrafish xenografts are generated by injecting a plurality of cancer cells into perivitelline space or the brain of zebrafish embryos or larvae (2-3dpf), wherein host zebrafish embryos or larvae are selected from wild-type strains and transgenic zebrafish lines, and the regressor tumor cells are tumor cell lines with engraftment rates lower than 30%, which increase upon immune suppression.

In step (b), the compounds added to the water fish are selected from chemicals, small molecules, drugs, antibodies, peptides, secreted proteins and mixtures thereof. The addition of the compounds can start in the injection day or 24 hours post-injection and compounds are renewed daily. Treated zebrafish xenografts will be compared to zebrafish xenografts treated with vehicle (controls).

In step (c), the engraftment rate is quantified, and the compounds are selected based on a significant increase of engraftment in relation to untreated controls (vehicle). In a preferred embodiment of the present invention, a significant increase of engraftment is an increase of at least 20% of engraftment, when compared to the controls.

In step (d), the tumor microenvironment modulation is performed by quantifying and characterizing the innate immune cell populations within the tumor. In a preferred embodiment of the present invention, the macrophages and neutrophils subtypes are characterized by using transgenic zebrafish lines for neutrophils and macrophages combined with cytokine reporters such as TNF-α and NFK-B.

In a forth aspect, the present invention relates to an in vivo method to use zebrafish patient-derived xenografts (zAvatars) as living biomarkers of the tumor microenvironment to select eligible patients for immunotherapy treatment, wherein the method comprises the steps of:

a) generating zebrafish patient-derived xenografts in control and immunocompromised hosts;

b) characterizing the generated tumor microenvironment by quantifying the innate immune infiltrate in different time points;

c) quantifying the engraftment rate; and

d) identifying eligible patients for immunotherapy based on their engraftment rate and pro-inflammatory (HOT) tumor microenvironment.

In step (a), the zebrafish xenografts are generated by injecting a plurality of patient cancer cells into perivitelline space or the brain of zebrafish embryos or larvae. In preferred embodiment of the present invention, the zebrafish embryos or larvae are selected from wild-type strains, transgenic zebrafish lines which label myeloid cells (for example, Tg(mpx:eGFP) Tg(mpeg1:mcherry) and Tg(mpeg1:mcherry, tnfa:GFP-F) and immunocompromised fish (for example, runx1w84x−/− and csfr1−/−, which lack neutrophils and macrophages, respectively or use chemicals that lead to immunosuppression).

In step (b), the generated tumor microenvironment is characterized by quantifying the innate immune infiltrate in different time points. In a preferred embodiment of the invention, the characterization occurs at 1 dpi (day post injection) and at the end of the assay. Therefore, the second time point depends on the duration of the assay, which may vary from 3 to 8 days.

In step (c), the engraftment rate is quantified by calculating the number of xenografts with a tumor at the end of the assay (3 to 8 dpi) divided by the total number of xenografts at the end of the assay.

At last, in step (d), patients whose zAvatars increase engraftment upon immunosuppression and whose TME at 1 dpi is highly inflammatory, e.g., tumors presenting HOT innate TME, are consequently more eligible for immunotherapy.

In summary, the methods herein disclosed in the present invention aim to identify innate immunomodulator compounds that can increase (for anti-cancer therapy) or reduce innate immunity (for transplantation, autoimmune diseases and chronic inflammatory diseases). It is also an objective of the present invention to find new biomarkers of immune response and stratify cancer patients for immunotherapy by identifying patients that have a tumor that generate a pro-inflammatory (HOT) tumor microenvironment.

### EXAMPLES

Zebrafish Xenografts Display Differential Engraftment Profiles

Using a zebrafish xenograft model (Fior et al., 2017), the engraftment efficiency of multiple human Breast and Colorectal Cancer (CRC) cell lines was investigated. At 4 days post injection (4 dpi), it was noticed that different cancer cell lines display distinct engraftment profiles in zebrafish xenografts.

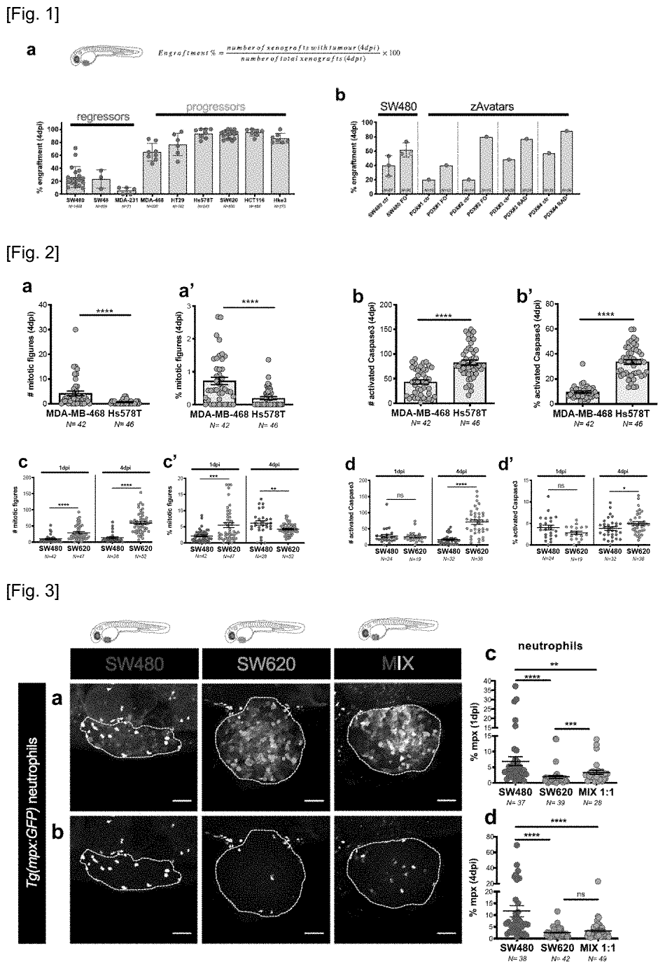

The engraftment is described as the frequency of xenografts that present a tumor (at least 30 tumor cells) at 4 dpi (FIG. 1a) and clearance as engraftment inhibition. It is observed that some cancer cell lines present a high engraftment rate—above 80% (progressors), while others engraft poorly with an average of engraftment rate of ˜20-30% (regressors).

Strikingly, it is possible to note differences in engraftment profiles between cancer cells derived from the same patient at different stages of tumor progression. While SW480 cells derived from the primary tumor present a regressor behavior, SW620 cells isolated from a lymph node metastasis six months later (Leibovitz et al., 1976 and Hewitt et al., 2000) show a progressor phenotype (FIG. 1a). These differences in engraftment rates between both tumor cells were also originally reported in mouse xenografts (Hewitt et al., 2000).

Importantly, engraftment/clearance capacity did not seem to correlate to proliferation potential or basal cell death. This is exemplified by the breast cancer cells Hs578T_progressors, which display a high engraftment rate (˜95% engraftment), despite their low proliferation and high level of apoptosis in comparison, for instance, with breast cancer MDA-MB-468, which display lower engraftment but are more proliferative and less apoptotic (FIG. 2 a-b′). Also, although SW620_progressors are highly proliferative compared with SW480_regressors, SW620_progressors present higher levels of apoptosis (FIG. 2 c-d′).

Moreover, paradoxically, SW480_regressors, upon chemo- (FOLFOX—FO) or radiotherapy (RAD), may increase their engraftment rate, and this can also be observed in patient-derived xenografts (zAvatars) (FIG. 1b). Given the fact that chemo/radio therapy may elicit an immunosuppressive effect, this could reduce the zebrafish host anti-tumor response, originally responsible for the regressor (clearance) behavior.

SW480 Regressor TME is Enriched in Innate Immune Cells.

To evaluate if regressors and progressors are able to generate different tumor ecosystems, the presence of neutrophils and macrophages in the tumors, the main innate immune cells present at this stage of development (2-6 days post fertilization dpf), was analyzed.

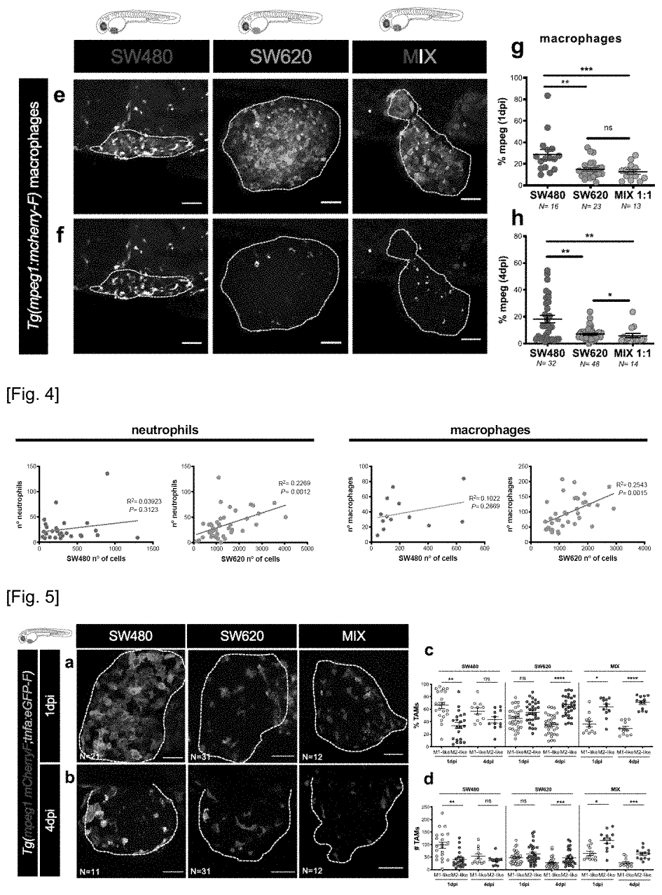

To this end, SW480, SW620, and MIX tumor cells were injected into Tg(mpx:eGFP) and Tg(mpeg1:mcherry-F) zebrafish hosts, which have neutrophils (FIG. 3a, b) and macrophages (FIG. 3e, f) labeled, respectively.

As early as 24 hpi (1 dpi), it was possible to detect a significant higher recruitment of neutrophils and macrophages to the SW480 tumors in comparison to SW620 (neutrophils P<0.0001, macrophages P=0.0011), a difference that was maintained and reinforced at 4 dpi (neutrophils P<0.0001, macrophages P=0.0089) (FIG. 3c, d, g, h).

Interestingly, MIX tumors showed a TME similar to SW620, with significant lower recruitment of neutrophils and macrophages than SW480 tumors (FIG. 3, SW480 vs MIX neutrophils P4 dpi<0.0001, macrophages P4 dpi=0.0025).

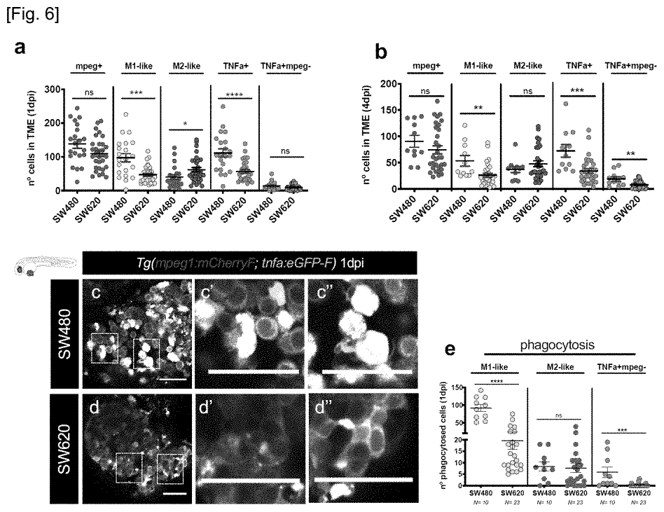

These results suggest that the presence of SW620 in the MIX is able to block the recruitment of immune cells toward the tumor. We next questioned whether immune cell recruitment was associated with the total number of tumor cells within the tumoral mass. Linear regression analysis of the tumor size vs immune cell counts suggests a weak correlation between tumor size and immune cell infiltrates in SW480 tumors, but moderate in SW620 tumors (FIG. 4).

SW480 and SW620 Tumors Modulate Zebrafish Macrophage Polarization.

In the TME, tumor-associated macrophages (TAMs) and neutrophils (TANs) can either adopt an anti-(M1/N1-like) or pro-tumoral (M2/N2-like) phenotype, known to be modulated by multiple tumor-derived signals (Nguyen-Chi M et al, 2015 and Galli et al, 2011). To investigate the polarization state of macrophages in both TMEs, SW480, and SW620 cells were injected into double transgenic animals Tg (mpeg1:mCherry-F; tnfa:eGFP-F) and each population was analyzed at 1 and 4 dpi (FIG. 5a, b and FIG. 6a, b).

Quantification of the immune cell populations showed that SW480_regressors are able to recruit a significantly higher number of inflammatory cells (TNFa positive cells and M1-like TNFa+mpeg+), than SW620_progressors, since 1 dpi (FIG. 6a, b, M1-like P1 dpi=0.0003; P4 dpi=0.001).

Moreover, when the proportions of M1-like (TNFa+) versus M2-like (TNFa−) macrophages were analyzed, it was observed that the SW480 TME presented ˜57% M1-like to 43% M2-like-macrophages at 4 dpi (FIG. 5c, d). In clear contrast, the TME of SW620_progressors cells presented a ratio of ˜35% M1-like to ˜65% M2-like macrophages (FIG. 5c). Interestingly, a progressive increase in M2-like-(TNFa−) macrophages in the TME of SW620 from 1 to 4 dpi was observed (FIG. 5c). This result suggests that SW620_progressor cells can polarize macrophages to a M2-like pro-tumoral state.

In addition, the MIX xenografts again show similar dynamics to SW620 xenografts (M2->M1-like macrophages), from 1 to 4 dpi (FIG. 5c, d). Moreover, as expected, a higher phagocytic activity (displayed by M1-like TNFa+/mpeg+ and TNFa+/mpeg−cells) was detected in SW480 TME than in SW620 (FIG. 6c-e, P<0.0001). In summary, these results show that human tumor cells are able to modulate the zebrafish TME toward a more anti or pro-tumoral state, through macrophage polarization and consequent phagocytic properties.

Zebrafish Innate Immune Cells Regulate SW480 Clearance.

The above results show that SW620_progressors protect SW480_regressors from being cleared and that SW480 cells are able to recruit more efficiently innate immune cells. Moreover, increasing amounts of SW620 in MIX xenografts correlate with increased engraftment of SW480 and the presence of SW620 seems sufficient to reduce immune cell infiltration. All together, these results suggest that innate immunity plays an active role in clearance/engraftment.

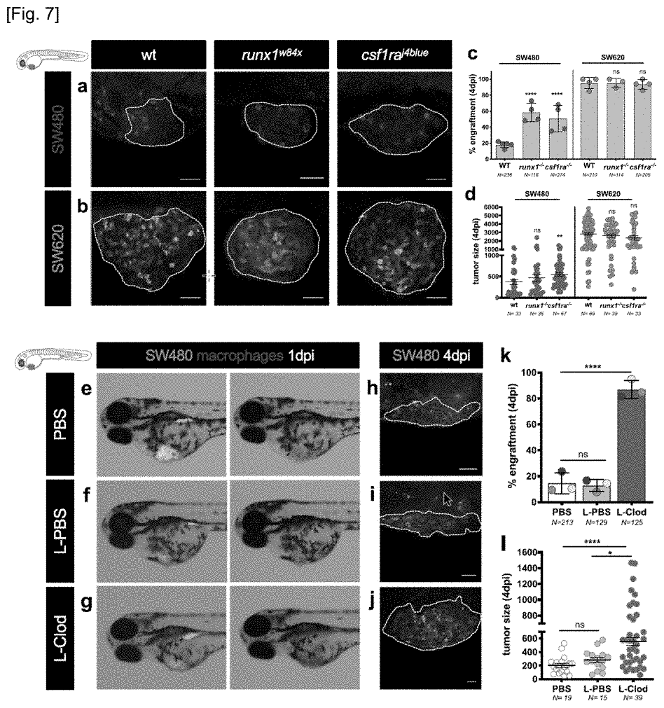

To directly test this, both CRC cell lines were injected into mutant zebrafish embryos that have either a transient downregulation of neutrophils (runx1w84x mutant) (Jin H et al., 2009) or of macrophages (M-CFS receptor/fms mutant csf1raj4blue panther) (Parichy D M et al., 2000) (FIG. 7a—b). The results show that runx1w84x and panther mutants present a significant increase in the engraftment of SW480 regressors cells (FIG. 7c). In runx1w84x mutants, a significant 3.2-fold increase of engraftment (P<0.0001) was observed, whereas in panther mutants it was observed a 2.8-fold of increase (P<0.0001) (FIG. 7c). In contrast, downregulation of neutrophils or macrophages had no significant impact on SW620_progressors' engraftment rate. Interestingly, quantification of tumor size in each background shows that SW480 regressors increase their size in panther mutants, which have reduced number of macrophages (FIG. 7d, P=0.0013). Overall, our results suggest that both myeloid cells play a crucial role in the SW480_regressors' clearance and that SW620_progressors are able to evade and/or suppress the host innate immune system.

Resident and Definitive Macrophages are Required for SW480 Clearance.

Recent studies have shown differential functions for resident macrophages and hematopoietic monocyte-derived macrophages in tumorigenesis (Loyher P L et al, 2018; Bowman R L et al, 2016 and Zhu Y et al., 2017). In 3 dpf zebrafish larvae, macrophages are distributed in several peripheral tissues, such as the brain, heart, retina, and muscle, and in the caudal hematopoietic tissue (CHT), a transient hematopoietic tissue (White R et al, 2013). In panther mutants (csf1raj4blue), it has been shown that there is an overall ˜40% reduction of the macrophage population and impairment of their migration. However, the tissue-resident macrophages (derived from the primitive and transient waves of hematopoiesis) show a stronger reduction (˜60%) than macrophages derived from the second-monocytic definitive wave (CHT-20%) (Herbomel P et al., 2001; Morales R A and Allende M L, 2019 and Pagan et al., 2015). The results observed in panther mutants thus reflect mostly the contribution of the resident macrophages (Morales R A and Allende M L, 2019). To further investigate the role of the different macrophages in tumor clearance, most macrophage population was depleted by using Liposome-Clodronate (L-clodronate), which targets macrophages regardless of their embryonic origin.

Strikingly, upon almost complete macrophage depletion (without affecting neutrophil numbers (Morales R A and Allende M L, 2019), see FIG. 7e—g), SW480 engraftment reaches almost 100% (FIG. 7k, P<0.0001), contrasting with the significant but less pronounced engraftment increase in panther mutants (˜60%, FIG. 7c). Moreover, quantification of the tumor size also shows that SW480 tumor size increases by almost 2-fold (FIG. 7h-j, l, L-PBS vs L-Clodro, P=0.02). In summary, our results highlight a major role for both tissue-resident and peripheral macrophages in tumor clearance.

Innate Immunoediting in Zebrafish Xenografts.

Next, it was analyzed if engrafted zebrafish SW480 tumors were undergoing innate immunoediting, and therefore, would be able to escape host innate immunity.

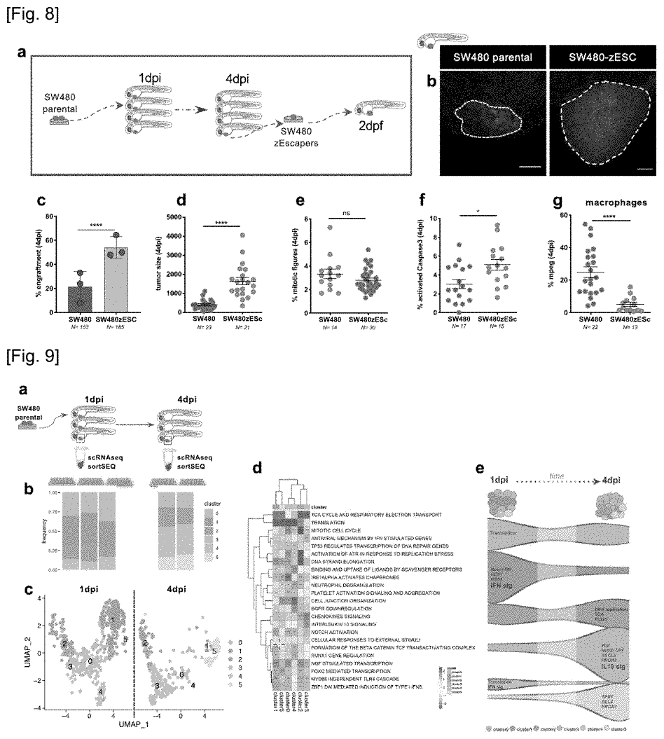

To this end, seven SW480 tumors were dissected at 4 dpi, from an experiment that yielded ˜12% engraftment. Dissected tumors were then expanded in vitro for three passages (FIG. 8a) and these (SW480zEscapers cells) were next injected into 2 dpf zebrafish embryos. Engraftment, tumor size, proliferation, apoptosis, and macrophage infiltration were quantified and compared to parental cells. Strikingly, SW480zEscapers engrafted much more efficiently (from an average of ˜20% in parental to ˜60% in SW480zEscapers, P<0.0001) (FIG. 8b, c) and tumor size increased in relation to parental tumors (FIG. 8d, P<0.0001).

Interestingly, it was not possible to detect a higher proliferation rate in these tumors (FIG. 8e) and apoptosis levels were slightly increased (FIG. 8f). Thus, these results reinforce the idea that proliferation and apoptosis are not the main drivers of engraftment/clearance. Importantly, the macrophage infiltrate was significantly reduced in these tumors (FIG. 8g, <0.0001). These results suggest that innate immunity plays a critical role in immunoediting cancer cells toward tumorigenesis.

Clearance and Expansion of Different SW480 Subclones.

To investigate the molecular alterations that might underlie the emergence of SW480 escapers (as well as the subclones that get cleared), it was performed single-cell transcriptomic profiling. SW480 parental cells (GFP transfected) was injected and then dissected tumors at 2 time-points for single-cell RNA-seq (scRNAseq): 1 dpi (where all subclones should be present) and at 4 dpi (where only the subclones that escape clearance are present) (FIG. 9a). Dissociated single cells were sorted by fluorescence activated cell sorting (FACS) into 384-well plates for scRNAseq SORT-seq35: 3 plates for the first timepoint and 2 plates for the second (FIG. 9a, b). Cells were pooled and clustered according to their gene expression profiles using Seurat36, resulting in six different cell clusters (cell states), which were visualized using the uniform manifold approximation and projection (UMAP) approach (Mcinnes et al., 2018) (FIG. 9b, c).

Comparing the clusters' frequency between 1 and 4 days, it was possible to follow how the various tumor clusters changed (FIG. 9) but also the dynamics of the signaling pathways (FIG. 9d). Interestingly, two cell clusters (1 and 4) almost disappear in just 3 days, whereas others maintain their frequency (0 and 2) but others clearly expand (3 and 5). These results suggest that some clusters were cleared (1 and 4), while others were able to evade innate immune detection and were therefore maintained (0, 2, 3, and 5) (FIG. 9e).

In cluster 1, whose frequency was strikingly reduced, enrichment pathway analysis showed the activation of innate immunerelated pathways as the interferon pathway (Myd88 independent TLR cascade and DNA-dependent activation of IFN-regulatory factors) as well as several inflammatory cytokines (e.g., CX3CL1, CXCL1) (FIG. 9d). These cytokines are known to act as chemoattractants for various immune cells; the large CX3CL1/fractalkine attracting T cells and monocytes (Bazan J F et al., 1997), whereas the small chemokine CXCL1 acts in particular to attract neutrophils during inflammation (Schumacher C et al., 1992).

Their increased expression in subclones that decrease frequency in the tumor might contribute to this clearance. In contrast, an enrichment of 11_10 immunosuppressive related signaling was observed in cluster 3 (which is expanded at 4 dpi), suggesting that IL10 signaling might protect SW480_zEscapers from clearance (FIG. 9d,).

In summary, our results show the clearance of specific regressors' subclones expressing IFN related signaling and Notch activation, as well as the expansion of subclones that express IL10 suppressive pathway with expansion of Wnt and secretory like “states” (cluster 3), as well as a putative “quiescent”-like progenitor state (cluster 5) (FIG. 9e).

Zebrafish Xenograft Model Reveals Macrophage Anti- or Pro-Tumoral Roles According Tumor Types

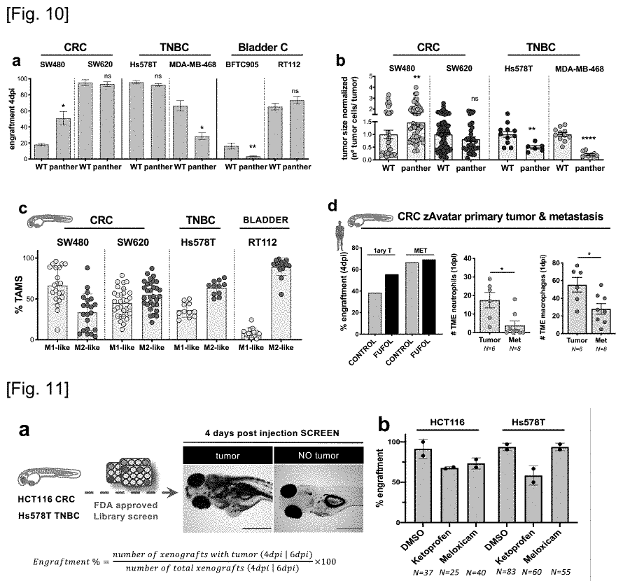

Using zebrafish mutants like Panther, where macrophages are impaired, the engraftment and tumor size of multiple Colorectal Cancer (CRC), Breast and Bladder cancer cell lines were investigated. FIG. 10a (which represents the number of xenografts analysed per condition minimal>60) shows that macrophages may have opposite impacts on engraftment and tumor size (b, wherein each dot represents a xenograft) depending on the tumor. Moreover, the modulation of the TME by different tumors (c) & zAvatars of CRC primary and metastasis show different engraftment rates (minimal of 20 xenografts per condition) and elicit differential recruitment of innate immune cells (d).

In Vivo Phenotypic Drug Screening to Find Molecules that Induce/Boost Clearance

Preliminary data from the FDA approved Library screen in colorectal and breast cancers are shown in FIG. 11a. Zebrafish xenografts were treated for 3 consecutive days with different compounds and respective DMSO control and screened according to the presence or absence of tumors, at 4 dpi (FIG. 11b). Until now, almost half of the library has been screened: 383 compounds in CRC (6 hits) and 283 in breast cancer (19 hits). As an example, NSAIDs Ketoprofen and Meloxican induced a ˜30% clearance of HCT116-CRC tumors whereas in Hs578T-TNBC, Ketoprofen induced a ˜45% clearance. Number of xenografts analyzed are depicted in the figure.

### CITATION LIST

### Non-Patent Literature

NPL1: Bazan, J. F. et al. A new class of membrane-bound chemokine with a CX3C motif. Nature 385, 640-644 (1997).
NPL2: Bejarano L, Jordāo MJC, Joyce J A. Therapeutic Targeting of the Tumor Microenvironment. Cancer Discov. 2021; 11(4):933-959.
NPL3: Blomberg O S, Spagnuolo L, de Visser K E. Immune regulation of metastasis: mechanistic insights and therapeutic opportunities. Dis Model Mech. 2018; 11(10):dmm036236.
NPL4: Bowman, R. L. et al. Macrophage ontogeny underlies differences in tumorspecific education in brain malignancies. Cell Rep. 17, 2445-2459 (2016).
NPL5: Cassetta L, Pollard J W. Targeting macrophages: Therapeutic approaches in cancer. Nat Rev Drug Discov. 2018; 17(12):887-904.
NPL6: Cerezo-Wallis D, Contreras-Alcalde M, Troulé K, et al. Midkine rewires the melanoma microenvironment toward a tolerogenic and immune-resistant state. Nat Med. 2020; 26(12): 1865-1877.
NPL7: Chapman A, del Ama L F, Ferguson J, Kamarashev J, Wellbrock C, Hurlstone A. Heterogeneous tumor subpopulations cooperate to drive invasion. Cell Rep. 2014; 8(3):688-695.
NPL8: Cortez-Retamozo V, Etzrodt M, Newton a., et al. Origins of tumor-associated macrophages and neutrophils. Proc Natl Acad Sci. 2012; 109(7):2491-2496.
NPL9: Costa, Estrada, Mendes, Fior. Zebrafish Avatars towards Personalized Medicine—A Comparative Review between Avatar Models. Cells. 2020; 9(2):293.
NPL10: Costa B, Ferreira S, Póvoa V, et al. Developments in zebrafish avatars as radiotherapy sensitivity reporters— towards personalized medicine. EBioMedicine. 2020; 51:1-12.
NPL11: DeNardo D G, Ruffell B. Macrophages as regulators of tumour immunity and immunotherapy. Nat Rev Immunol. 2019; 19(6):369-382.
NPL12: Fazio M, Ablain J, Chuan Y, Langenau D M, Zon L I. Zebrafish patient avatars in cancer biology and precision cancer therapy. Nat Rev Cancer. 2020; 20(5):263-273.
NPL13: Fazio M, Zon L I. Fishing for answers in precision cancer medicine. Proc Natl Acad Sci USA. 2017; 114(39):10306-10308.
NPL14: Fior R, POvoa V, Mendes R V., et al. Single-cell functional and chemosensitive profiling of combinatorial colorectal therapy in zebrafish xenografts. Proc Natl Acad Sci. 2017; 114(39):201618389.
NPL15: Gajewski T F, Schreiber H, Fu Y-X. Innate and adaptive immune cells in the tumor microenvironment. Nat Immunol. 2013; 14:1014.
NPL16: Galli, S. J., Borregaard, N. & Wynn, T. A. Phenotypic and functional plasticity of cells of innate immunity: macrophages, mast cells and neutrophils. Nat. Publ. Gr. 12, 1035-1044 (2011).
NPL17: Gentles A J, Newman A M, Liu C L, et al. The prognostic landscape of genes and infiltrating immune cells across human cancers. Nat Med. 2015; 21(8):938-945.
NPL18: Grönholm M, Feodoroff M, Antignani G, Martins B, Hamdan F, Cerullo V. Patient-Derived Organoids for Precision Cancer Immunotherapy. Cancer Res. 2021; 81(12):3149 LP—3155.
NPL19: Herbomel, P., Thisse, B. & Thisse, C. Zebrafish early macrophages colonize cephalic mesenchyme and developing brain, retina, and epidermis through a M-CSF receptor-dependent invasive process. Dev. Biol. 238, 274-288 (2001).
NPL20: Hewitt R E, McMarlin A, Kleiner D, et al. Validation of a model of colon cancer progression. J Pathol. 2000; 192(4):446-454.
NPL21: Jin, H. et al. Definitive hematopoietic stem/progenitor cells manifest distinct differentiation output in the zebrafish VDA and PBI. Development 136, 1397 (2009).
NPL22: Lam S, Wu Y, Vega V, et al. Conservation of gene expression signatures between zebrafish and human liver tumors and tumor progression. Nat Biotechnol. 2006; 24(1):73-75.
NPL23: Leibovitz A, Stinson J C, McCombs W B, McCoy C E, Mazur K C, Mabry N D. Classification of Human Colorectal Adenocarcinoma Cell Lines. Cancer Res. 1976; 36(12):4562-4569.
NPL24: Liu Y, Cao X. Immunosuppressive cells in tumor immune escape and metastasis. J Mol Med. 2016; 94(5):509-522.
NPL25: Liu Y, Zeng G. Cancer and innate immune system interactions: Translational potentials for cancer immunotherapy. J Immunother. 2012; 35(4):299-308.
NPL26: Loyher, P. L. et al. Macrophages of distinct origins contribute to tumor development in the lung. J. Exp. Med. 215, 1-18 (2018).
NPL27: McInnes, L., Healy, J., Saul, N. & GroBBerger, L. UMAP: uniform manifold approximation and projection. J. Open Source Softw. 3, 861 (2018).
NPL28: Morales, R. A. & Allende, M. L. Peripheral macrophages promote tissue regeneration in zebrafish by fine-tuning the inflammatory response. Front. Immunol. 10, 1-14 (2019).
NPL29: Nguyen-Chi, M. et al. Identification of polarized macrophage subsets in zebrafish. Elife 4, 1-14 (2015).
NPL30: Oh B Y, Lee W Y, Jung S, et al. Correlation between tumor engraftment in patient-derived xenograft models and clinical outcomes in colorectal cancer patients. Oncotarget. 2015; 6(18):16059-16068.
NPL31: Pagan, A. J. et al. Myeloid growth factors promote resistance to mycobacterial infection by curtailing granuloma necrosis through macrophage replenishment. Cell Host Microbe 18, 15-26 (2015).
NPL32: Parichy, D. M., Ransom, D. G., Paw, B., Zon, L. I. & Johnson, S. L. An orthologue of the kit-related gene fms is required for development of neural crest-derived xanthophores and a subpopulation of adult melanocytes in the zebrafish, Danio rerio. Development 127, 3031-3044 (2000).
NPL33: Póvoa V, Rebelo de Almeida C, Maia-Gil M, et al. Innate immune evasion revealed in a colorectal zebrafish xenograft model. Nat Commun. 2021; 12(1):1156.
NPL34: Rebelo de Almeida C, Mendes R V, Pezzarossa A, et al. Zebrafish xenografts as a fast screening platform for bevacizumab cancer therapy. Commun Biol. 2020; 3(1).
NPL35: Rennekamp A J, Peterson R T. 15 Years of Zebrafish Chemical Screening. Curr Opin Chem Biol. 2015; 24:58-70.
NPL36: Renshaw S a., Trede N S. A model 450 million years in the making: zebrafish and vertebrate immunity. Dis Model Mech. 2012; 5(1):38-47.
NPL37: Roh-Johnson M, Shah A N, Stonick J A, et al. Macrophage-Dependent Cytoplasmic Transfer during Melanoma Invasion In Vivo. Dev Cell. 2017; 43(5):549-562.e6.
NPL38: Schreiber R D, Old L J, Smyth M J. Cancer Immunoediting: Integrating Immunity's Roles in Cancer Suppression and Promotion. Science (80-). 2011; 331(6024):1565-1570.
NPL39: Schumacher, C., Clark-Lewis, I., Baggiolini, M. & Moser, B. High- and low-affinity binding of GRO alpha and neutrophil-activating peptide 2 to interleukin 8 receptors on human neutrophils. Proc. Natl Acad. Sci. USA 89, 10542 LP-10510546 (1992).
NPL408: Van Overmeire E, Laoui D, Keirsse J, Van Ginderachter J A, Sarukhan A. Mechanisms driving macrophage diversity and specialization in distinct tumor microenvironments and parallelisms with other tissues. Front Immunol. 2014; 5(March): 1-16.
NPL41: Varanda A B, Martins-Logrado A, Ferreira M G, Fior R. Zebrafish xenografts unveil sensitivity to olaparib beyond BRCA status. Cancers (Basel). 2020; 12(7):1-20.
NPL42: Vladimer G I, Snijder B, Krall N, et al. Global survey of the immunomodulatory potential of common drugs. Nat Chem Biol. 2017; 13(6):681-690.
NPL43: White R, Rose K, Zon L. Zebrafish cancer: the state of the art and the path forward. Nat Rev Cancer. 2013; 13(9):624-636.
NPL44: Wyatt R A, Trieu N P V, Crawford B D. Zebrafish xenograft: An evolutionary experiment in tumour biology. Genes (Basel). 2017; 8(9).
NPL45: Zhu, Y. et al. Correction: Tissue-resident macrophages in pancreatic ductal adenocarcinoma originate from embryonic hematopoiesis and promote tumor progression. Immunity 47, 597 (2017).

1. In vivo method to find mechanisms of innate immune evasion/suppression and biomarkers of the immune response, characterized by comprising the steps of:
a. generating zebrafish xenografts;
b. dissecting the tumors from zebrafish xenografts collected in different time points;
c. quantifying the engraftment rate;
d. analyzing and comparing human vs zebrafish cells to identify human candidate genes and zebrafish interacting partners;
e. testing the functional role of the human candidate genes identified in step (d); and
f. validating the clinical relevance of the human candidate genes as biomarkers of the immune response.

2. Method, according to claim 1, wherein, in step (a), the zebrafish xenografts are generated by injecting a plurality of cancer cells into perivitelline space or the brain of host zebrafish embryos or larvae.

3. Method, according to claim 1, wherein host zebrafish embryos or larvae are selected from wild-type strains and transgenic zebrafish lines.

4. Method, according to claim 1, wherein, in step (b), the tumors from zebrafish xenografts are dissected in two different time points.

5. Method, according to claim 1, wherein, the different time points are at 1 dpi and at the end of the assay, which varies from 4 to 8 dpi.

6. Method, according to claim 1, wherein a pool of approximately 100 tumor cells is collected in each time point.

7. Method, according to claim 1, wherein the engraftment rate is quantified by calculating the number of xenografts with a tumor at the end of the assay divided by the total number of xenografts at the end of the assay.

8. Method, according to claim 1, wherein, in step (d), the human and zebrafish cells are analyzed and compared by bioinformatic analysis of results obtained by genomics, transcriptomics, proteomics and metabolomics.

9. Method, according to claim 1, wherein, in step (e), the human candidate genes are tested by performing gain and loss of function experiments.

10. Method, according to claim 1, wherein, in step (f), the human candidate genes are validated by bioinformatic analysis and comparison to omics databases.

11. In vivo method to identify innate immunomodulators to boost tumor clearance using zebrafish xenografts, characterized by comprising the steps of:
a) generating zebrafish xenograft using progressor tumor cells;
b) adding compounds to the water fish;
c) quantifying the engraftment rate and selecting compounds based on a significant reduction of engraftment; and
d) validating the compounds in vivo using zebrafish transgenic hosts and immunocompromised zebrafish to confirm modulation through tumor microenvironment.

12. Method, according to claim 11, wherein, in step (a), the zebrafish xenografts are generated by injecting a plurality of cancer cells into perivitelline space or the brain of host zebrafish embryos or larvae.

13. Method, according to claim 11, wherein host zebrafish embryos or larvae are selected from wild-type strains and transgenic zebrafish lines.

14. Method, according to claim 11, wherein the progressor tumor cells are tumor cell lines with engraftment rates higher than 70%.

15. Method, according to claim 11, wherein, in step (b), the compounds are selected from chemicals, small molecules, drugs, antibodies, peptides, secreted proteins and mixtures thereof.

16. Method, according to claim 11, wherein the compounds are added in the injection day or 24 hours post injection and daily renewed.

17. Method, according to claim 11, wherein, in step (c), a significant reduction of engraftment is a reduction of at least 20% of engraftment, when compared to untreated controls.

18. Method, according to claim 11, wherein, in step (d), the immunocompromised zebrafish are zebrafish mutants, chemicals, morpholinos and mixtures thereof.

19. In vivo method to identify innate immune suppressor compounds for autoimmune diseases, organ transplantation or for conditions marked by uncontrolled inflammation, characterized by comprising the steps of:
a) generating zebrafish xenograft using regressor tumor cells;
b) adding compounds to the water fish;
c) quantifying the engraftment rate and selecting the compounds based on a significant increase of engraftment; and
d) characterizing the tumor microenvironment modulation upon treatment with the selected compounds.

20. Method, according to claim 19, wherein, in step (a), the zebrafish xenografts are generated by injecting a plurality of cancer cells into perivitelline space or the brain of host zebrafish embryos or larvae.

21. Method, according to claim 20, wherein host zebrafish embryos or larvae are selected from wild-type strains and transgenic zebrafish lines.

22. Method, according to claim 19, wherein regressor tumor cells are tumor cell lines with engraftment rates lower than 30%, which increase upon immune suppression.

23. Method, according to claim 19, wherein, in step (b), the compounds are selected from chemicals, small molecules, drugs, antibodies, peptides, secreted proteins and mixtures thereof.

24. Method, according to claim 19, wherein the compounds are added in the injection day or 24 hours post injection and daily renewed.

25. Method, according to claim 19, wherein, in step (c), a significant increase of engraftment is an increase of at least 20% of engraftment, when compared to untreated controls.

26. In vivo method to use zebrafish patient-derived xenografts as living biomarkers of the tumor microenvironment to select eligible patients for immunotherapy treatment, characterized by comprising the steps of:
a) generating zebrafish patient-derived xenografts in control and immunocompromised hosts;
b) characterizing the generated tumor microenvironment by quantifying the innate immune infiltrate in different time points;
c) quantifying the engraftment rate; and
d) identifying eligible patients for immunotherapy based on their engraftment rate and pro-inflammatory (HOT) tumor microenvironment.

27. Method, according to claim 26, wherein, in step (a), the zebrafish patient-derived xenografts are generated by injecting a plurality of patient cancer cells into perivitelline space or the brain of zebrafish embryos or larvae.

28. Method, according to claim 27, wherein zebrafish embryos or larvae are selected from wild-type strains, transgenic zebrafish lines which label myeloid cells and, immunocompromised fish.

29. Method, according to claim 26, wherein, in step (b), the different time points are at 1 dpi and at the end of the assay, which varies from 3 to 8 dpi.

30. Method, according to claim 26, wherein, in step (c), the engraftment rate is quantified by calculating the number of xenografts with a tumor at the end of the assay divided by the total number of xenografts at end of the assay.

31. Method, according to claim 26, wherein, in step (d), eligible patients for immunotherapy are selected from those whose zebrafish xenografts increase the engraftment upon immunosuppression and whose tumor microenvironment at 1 dpi is highly inflammatory.
